(19) United States
(12) Patent Application Publication
(10) Pub. No.: US 2005/0136108 A1
(43) Pub. Date: Jun. 23, 2005

(54) Stepwise delivery of topiramate over prolonged period of time

(76) Inventors: Noymi V. Yam, Sunnyvale, CA (US); Padmaja Shivanand, Los Altos, CA (US); Rhea Kimbel, Mountain View, CA (US); Clark P. Allphin, Mountain View, CA (US); Patrick S.L. Wong, Burlingame, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/920,934

(22) Filed: Aug. 18, 2004

Related U.S. Application Data
(60) Provisional application No. 60/497,162, filed on Aug. 22, 2003.

(51) Int. Cl. A61K 9/22

(52) U.S. Cl. 424/468

(57) ABSTRACT

Compositions and dosage forms for enhanced dispersion of topiramate in a controlled release dosage form released from the dosage form as a dry or substantially dry erodible solid over a prolonged period of time at a stepwise increasing rate of release are described.

### CROSS REFERENCE TO RELATED APPLICATION

This application claims priority from U.S. Provisional Application Ser. No. 60/497,162, filed Aug. 22, 2003, the contents of which are hereby incorporated by reference in their entirety.

### FIELD OF THE INVENTION

This invention pertains to the controlled delivery of pharmaceutical agents and methods, dosage forms and devices therefor. In particular, the invention is directed to formulations, dosage forms and devices for enhancing controlled delivery of topiramate by use of a composition that increases the dispersion of the pharmaceutical agent. The present invention provides a means for delivering high doses of the lowly soluble drug topiramate at a stepwise, increasing rate from a solid dosage form system that is convenient to swallow.

### BACKGROUND OF THE INVENTION

The art is replete with descriptions of dosage forms for the controlled release of pharmaceutical agents. While a variety of sustained release dosage forms for delivering certain drugs may be known, not every drug may be suitably delivered from those dosage forms because of solubility, metabolic processes, absorption and other physical, chemical and physiological parameters that may be unique to the drug and mode of delivery.

Dosage forms that incorporate lowly soluble drugs with poor dissolution rates at high drug loading provide a major challenge for controlled release delivery technology. Such systems tend to be of such large size that patients are unwilling or unable to swallow them.

Topiramate is indicated as an antiepileptic drug. Topiramate is a white crystalline powder, which is soluble in alkaline solutions containing sodium hydroxide or sodium phosphate, soluble in acetone, dimethylsulfoxide and ethanol. However, the solubility of topiramate in water is only about 9.8 mg/ml and the rate of dissolution is poor. Topiramate is not extensively metabolized and-is excreted largely through the urine. Physicians' Desk Reference, Thompson Healthcare, 56^{th} Ed., pp. 2590-2591 (2002).

Topiramate is currently marketed as Topamax® by Ortho-McNeil Pharmaceutical, Inc., Raritan, N.J., and disclosed more fully in U.S. Pat. No. 4,513,006.

Topiramate pharmacokinetics is linear producing a dose proportional increase in blood plasma concentration levels and there is no evidence of tolerance. Topamaxe is traditionally dosed at 400 mg/day with two divided dosages. Doses above 400 mg/day (600 mg/day, 800 mg/day and 1000 mg/day) have been tested, but have not shown significantly improved responses. Lower doses than 400 mg/day (e.g. 200 mg/day) demonstrated inconsistent effects. However, the lower doses may be appropriate for pediatric use or yet to be determined indications. Physicians' Desk Reference, Thompson Healthcare, 56^{th} Ed., pp. 2590-2595 (2002).

The low solubility and poor dissolution characteristics of topiramate along with high daily dosing requirements do not motivate towards a once-a-day formulation, even in an osmotic delivery system. Conventional osmotic systems manage to deliver low solubility drugs by incorporating surfactants into the drug composition, sometimes at high percentages of the total drug composition, to increase solubility. However, this does not support a high drug loading system that is easily swallowed. These conventional osmotic systems release the drug as a solution or suspension through a small orifice in the dosage form and can achieve high bioavailability. There remains a need for high drug loading in a once-a-day system able to achieve the same high level of bioavailability. The present invention achieves this result by delivering the drug from an osmotic dosage form as an erodible solid composition released through a large orifice at a controlled rate from the dosage form without the need for any surfactant in the composition.

Conventional devices in which a drug composition is delivered as a slurry, suspension or solution from a small exit orifice by the action of an expandable layer are described in U.S. Pat. Nos. 5,633,011; 5,190,765; 5,252,338; 5,620,705; 4,931,285; 5,006,346; 5,024,842; and 5,160,743. Typical devices include a tablet comprising an expandable push layer and a drug layer, which tablet is surrounded by a semipermeable membrane having a delivery orifice. In certain instances, the tablet is provided with a subcoat to delay release of the drug composition to the environment of use.

Devices in which a drug composition is released in a dry state from a large exit orifice by the action of an expandable layer are described in U.S. Pat. Nos. 4,892,778, 4,915,949 and 4,940,465 and 5,023,088. Those references describe a dispenser for delivering a beneficial agent to an environment of use that includes a semipermeable wall containing a layer of expandable material that pushes a dry drug layer composition out of the compartment formed by the wall. The exit orifice in the device is substantially the same diameter as the inner diameter of the compartment formed by the wall. In such devices, a substantial area of the drug layer composition is exposed to the environment of use leading to release performance that can be subject to the stirring conditions in such environment.

While previous dosage forms delivering a drug composition to the environment of use in the dry state through a large delivery orifice may provide suitable release of drug over a prolonged period of time, the exposure of the drug layer to the variably turbulent fluid environment of use such as the upper gastrointestinal tract may result in agitation-dependent release of drug that in some circumstances is difficult to control. Moreover, such dosage forms delivering in the dry state into a semisolid environment lacking sufficient volumes of bulk water such as in the lower colonic environment of the gastrointestinal tract may have difficulty solubilizing the dry drug composition into the environment as the high solids content composition tends to adhere to the dosage form at the site of the large orifice. Accordingly, the present invention seeks to avoid these disadvantages to minimize effects of localized stirring conditions on delivery performance.

Other similar devices have delivered drug by expelling discrete drug containing tablets at a controlled rate over time. U.S. Pat. Nos. 5,938,654; 4,957,494; 5,023,088; 5,110,597; 5,340,590; 4,824,675; and 5,391,381.

Other devices attempt to deliver low solubility drugs by incorporating liquid drug formulations that are released at a controlled rate over time. These devices are disclosed in U.S. Pat. Nos. 4,111,201; 5,324,280; 5,413,672; and 6,174,547. However, such liquid osmotic delivery systems are limited in the concentration of drug in the liquid formulation and hence, the drug loading available, leading to delivery systems that can be of an unacceptably large size or number for therapeutic purposes. Still other delivery systems utilize a liquid carrier to deliver tiny time pills suspended within the liquid carrier. Such devices are disclosed in U.S. Pat. Nos. 4,853,229 and 4,961,932. These suspensions require that the therapeutic dose of pharmaceutical agent be dispensed by volume with measuring devices such as graduated cylinders or measuring spoons, a dispensing process that can be messy and inconvenient for the patient to administer.

Still others deliver by various means of delaying release of a drug For example, U.S. Pat. No. 5,536,507 describes a three component pharmaceutical formulation that utilizes, inter alia, a pH sensitive polymer and optionally an osmotic agent that will swell in the higher pH regions of the lower portion of the small intestine and the large intestine to release drug in those environments. Additional components of the dosage form include a delayed release coating and an enteric coating to provide a dosage form that releases very little, if any, of the drug in the stomach, a relatively minimal amount in the small intestine and reportedly about 85% or more in the large intestine. Such a dosage form provides for a widely varying time-release of drug after administration that may not begin for 1-3 hours until the dosage form has passed from the stomach and an additional 3 hours or more for the dosage form to pass into the large intestine.

The conventional dosage forms described above deliver therapeutic agents at an approximately zero order rate of release. Recently, dosage forms have been disclosed for delivering certain drugs at approximately ascending rates of release such as ALZA Corporation's Concerta® methylphenidate product. PCT Published Application Nos. U.S. 99/11920 (WO 9/62496); U.S. 97/13816 (WO 98/06380); and U.S. 97/16599 (WO 98/14168). Such disclosed dosage forms involve the use of multiple drug layers with sequentially increasing concentrations of drug in each drug layer to produce the increasing delivery rate of drug over time. While such multi-layer tablet constructions represent a significant advancement to the art, these devices also have limited capability of delivering lowly soluble pharmaceutical agents, particularly those associated with relatively large doses of such agents, in a size that is acceptable for patients to swallow.

An aspect of delivery of topiramate described herein is that the administration of high dosages of drug may require drug loading in the drug compositions and dosage forms being administered to be in the range of 20% to 90% of the overall weight of the composition or dosage form and preferably about 40% of the core. Such loading requirements may present problems in formulating compositions and fabricating dosage forms and devices that are suitable for oral administration and can be swallowed without undue difficulty. Loading requirements may present problems when formulating dosage forms that are to be administered a limited number of times per day, such as for once-a-day dosing, with a goal of uniform release of active agent over a prolonged period of time.

While a variety of sustained release dosage forms for delivering certain drugs exhibiting short half-life may be known, not every drug may be suitably delivered from those dosage forms because of solubility, metabolic processes, absorption and other physical, chemical and physiological parameters that may be unique to the drug and the mode of delivery.

Thus, there remains a critical need for a means to deliver high doses of topiramate at various delivery patterns in dosage forms that are feasible and convenient for patients to swallow. The need includes effective dosing methods, dosage forms and devices that will permit the controlled release of topiramate over a prolonged period of time in order to increase the time between dosing, preferably twice a day and most preferably to obtain a once-a-day dosing regimen. Such dosage forms should preferably have the option of delivering at an approximately zero order rate of release, ascending or other hybrid delivery rate pattern appropriate for the therapeutic agent being delivered.

### SUMMARY OF THE INVENTION

The present invention unexpectedly provides a drug composition for both a dosage form and method for controlled delivery of high doses of topiramate over an extended period of time, preferably providing once-a-day administration. This is accomplished through the use of three primary components in the drug composition: topiramate, a structural polymer carrier, and a disintegrant without a solubilizing surfactant. Furthermore, the present invention is characterized by incorporation of this composition into an osmotic delivery dosage form wherein the dry erodible composition is released through a large orifice in the dosage form at a controlled rate to the environment of use where it erodes to deliver the active agent. The invention is further characterized by two drug layer compositions within the dosage form that release consecutively to produce a stepwise or ascending rate of release from the dosage form depending upon the type and configuration of the osmotic delivery device and layers.

Conventional osmotic delivery involves the use of surfactants to achieve an increased degree of drug solubilization. The present invention offers a different approach for delivering moderate to low solubility drugs that have poor dissolution rate kinetics. The characteristic of this approach is that the system provides dispersion of the active agent as an alternative to solubilization. The proposed formulation employs primarily the drug, a carrier, and a disintegrant that will provide the dispersion of the active agent.

The present invention is directed to a novel drug core composition for an osmotic dosage form to provide therapeutic effects over 24 hours utilizing a single convenient solid oral dosage form. The dosage form releases topiramate at a stepwise increase rate of release for up to about 24 hours, preferably with once-a-day administration using a drug core composition.

The structural polymers Polyox® N80; Polyox® N10; Maltrin M100; polyvinylpyrrolidone (PVP) 12PF; PVP K2932; Klucel EF and Kollidon VA64 were surprisingly found to provide the optimal functionality for prolonged controlled delivery of high doses of topiramate from an osmotic delivery system, and most preferably Polyox® N80.

The present invention involves release of topiramate in high doses through providing increased dispersion to achieve high levels of absorption in-vivo without the use of a solubilizing surfactant.

The drug composition of the present invention may further allow the bioavailability of the therapeutic agent to be enhanced through increased absorption of topiramate in the gastrointestinal tract, especially in the colonic region, that otherwise would not be absorbed due to the lack of sufficient bulk water to sufficiently solubilize the drug.

The present invention is preferably incorporated into an osmotic dosage form having a semipermeable membrane enveloping a bi-layer or multi-layer core containing at least a first drug composition layer, containing a therapeutic agent and excipients, and a second expandable layer referred to as the push layer containing osmotic agents and no therapeutic agent. At least one orifice is drilled through the membrane on the drug-layer end of the tablet for allowing release of the active agent to the environment.

In the present invention the drug composition is released as a dry or substantially dry erodible composition from a large diameter orifice in the osmotic dosage form.

In the aqueous environment of the gastrointestinal (GI) tract, water is imbibed through the semipermeable membrane at a controlled rate. This causes the push layer to swell and expand against the dry drug layer composition, which is pushed out through the large orifice in a solid, dry or substantially dry state. The drug layer composition exits the system through the orifice in the membrane over a prolonged period of time as water from the gastrointestinal tract is imbibed into the delivery system. The dry drug layer composition released from the dosage form is eroded in the gastrointestinal tract to disperse and deliver the active agent to the environment. At the completion of drug release, the biologically inert components of the delivery system are eliminated as a tablet shell.

In one aspect, the present invention comprises a drug composition containing topiramate in a controlled release dosage form adapted to release as a dry or substantially dry erodible composition over a prolonged period of time at a controlled rate of release.

In yet another aspect, the invention comprises a method of treating a condition in a subject responsive to administration of topiramate, which comprises orally administering to the subject an osmotic dosage form having a drug core composition adapted to release topiramate at a controlled stepwise increasing rate of release over a prolonged period of time. Preferably, the dosage form is administered orally, once a day.

In still another aspect, the invention comprises a drug core composition for an osmotic dosage form comprising a wall defining a compartment, the wall having at least one exit orifice formed or formable therein and at least a portion of the wall being semipermeable; an expandable layer located within the compartment remote from the exit orifice and in fluid communication with the semipermeable portion of the wall; and at least one drug core composition layer located within the compartment adjacent the exit orifice, the drug layer composition comprising topiramate and a structural polymer carrier without a surfactant.

The prior art did not appreciate that high doses of topiramate could be made into a single controlled release dosage form or into a solid therapeutic composition as claimed herein that provides efficacious therapy over 24 hours with once-a-day administration. The prior art did not appreciate that a solid dosage form and a therapeutic composition can be made available comprising topiramate, a structural polymer carrier and an optional disintegrant without a surfactant.

The drug core composition of the present invention embodies a combination of topiramate and structural polymer, which structural polymer is present to provide a dual role of imparting structural integrity to the solid drug core in the dry state and of providing disintegrating properties during erosion and in the wet state during the operation of the dosage form. The structural viscosity develops as a result of the formation of a functional hydrogel while the delivery system is in operation. The structural polymer comprises a hydrophilic polar polymer that freely interacts with polar molecules of water to form the structurally viscous mass bearing sufficient viscosity necessary to effectively suspend and conduct the dispersed and dissolved drug from the dosage form.

The above presentation dictates the critical need for a drug core composition for a solid pharmaceutical dosage form and for a therapeutic composition that overcomes the shortcomings of conventional solid osmotic dosage forms, including tablets and capsules. These conventional dosage forms do not provide for optimal dose-regulated drug therapy over an extended period of time with high doses of lowly soluble drugs.

In still another aspect, the invention comprises a dosage form comprising a wall defining a compartment, the wall having an exit orifice formed or formable therein and at least a portion of the wall being semipermeable; an expandable layer located within the compartment remote from the exit orifice and in fluid communication with the semipermeable portion of the wall; and a drug layer located within the compartment adjacent the exit orifice, the drug layer comprising topiramate. The dosage form may optionally comprise a flow-promoting layer between the wall and the drug layer.

In another aspect, the invention comprises a method of treating a condition responsive to administration of topiramate or a pharmaceutically acceptable acid addition salt thereof, which comprises administering the compound to provide a steady state plasma concentration of the compound of between 5 ng/ml and 5000 ng/ml with the proviso that during the 24 hour period after administration of the dosage form the quotient formed by [Cₘₐₓ−Cₘᵢₙ]/C_{avg} is 3 or less.

### BRIEF DESCRIPTION OF THE FIGURES

FIGS. 1A and 1 B illustrate an embodiment of a dosage form of this invention having a single drug composition layer, FIG. 1A illustrating the dosage form prior to administration to a subject and FIG. 1B illustrating the dosage form at a period of time after administration to a subject;

FIG. 2 illustrates an embodiment of a dosage form of the present invention having two drug composition layers.

FIG. 3 illustrates a release profile (release rate as a function of time) of the active agent topiramate from a representative dosage form having the general characteristics of FIG. 2, after multiple dosings;

FIG. 4 illustrates a release profile (release rate as a function of time) of the active agent topiramate from a representative dosage form having the general characteristics of FIG. 1A, formed with an orifice of 145 mils and containing 100 mg of topiramate with 60% topiramate in the drug layer;

FIG. 5 illustrates release rate profiles for the active agent topiramate from a representative dosage form having the general characteristics of FIG. 2 of the present invention with various concentrations of drug in the first drug layer and the second drug layer by weight percent, formed with an orifice of 145 mils and containing 100 mg of topiramate. Example 2 herein describes the formulation resulting in the release rate of FIG. 5C and Example 3 herein describes the formulation resulting in the release rate of FIG. 5D.

FIG. 6 illustrates the release rate profile for the active agent topiramate from a representative dosage form of the present invention having the general characteristics of FIG. 2 containing 200 mg of topiramate as described in Example 4, herein.

### DETAILED DESCRIPTION OF THE INVENTION

The present invention is best understood by reference to the following definitions, the drawings and exemplary disclosure provided herein.

Definitions

By “dosage form” is meant a pharmaceutical composition or device comprising an active pharmaceutical agent, such as topiramate or a pharmaceutically-acceptable acid addition salt thereof and a structural polymer without a solubilizing surfactant and the composition or device optionally containing inactive ingredients, i.e., pharmaceutically acceptable excipients such as disintegrants, binders, diluents, lubricants, stabilizers, antioxidants, osmotic agents, colorants, plasticizers, coatings and the like, that are used to manufacture and deliver active pharmaceutical agents.

By “active agent”, “pharmaceutical agent”, “therapeutic agent” or “drug” is meant topiramate or an agent, drug, or compound having the therapeutic characteristics of topiramate or a pharmaceutically acceptable acid addition salt thereof.

By “pharmaceutically-acceptable acid addition salt” or “pharmaceutically acceptable salt”, which are used interchangeably herein, are meant those salts in which the anion does not contribute significantly to the toxicity or pharmacological activity of the salt, and, as such, they are the pharmacological equivalents of the bases of the compound. Examples of pharmaceutically acceptable acids that are useful for the purposes of salt formation include but are not limited to hydrochloric, hydrobromic, hydroiodic, citric, succinic, tartaric, maleic, acetic, benzoic, mandelic, phosphoric, nitric, palmitic, and others.

By “lowly soluble” and “low solubility” is meant that the neat therapeutic agent in the absence of solubilizing surfactants exhibits solubility in water of no more than 100 milligrams per milliliter. Aqueous solubility is determined by adding the therapeutic agent to stirred or agitated water maintained in a constant temperature bath at a temperature of 37 degrees centigrade until no more agent dissolves. The resulting solution saturated with active agent is then filtered, typically under pressure through a 0.8-micron Millipore filter, and the concentration in solution is measured by any appropriate analytical method including gravimetric, ultraviolet spectrophometry, chromatography, and the like.

By “low dissolution rate” or “poor dissolution rate” is meant that the neat therapeutic agent in the absence of solubilizing surfactants exhibits dissolution rates in water of less than 0.1 mg/min/cm2. Intrinsic dissolution is a method that utilizes a compressed disc of known area (a constant surface), effectively eliminating surface area and surface electrical charges as dissolution variables. The dissolution rate obtained by this method is termed the intrinsic dissolution rate and is characteristic of each solid compound in a given solvent under the fixed experimental conditions. The value is generally expressed as mg dissolved per minute per centimeters squared and is useful in predicting probable absorption problems due to dissolution rate. Intrinsic rates of >1.0 mg/min/cm2 have negligible problems with dissolution rate limitations, but rates <0.1 mg/min/cm2 suggest problems with dissolution rate limitations. See USP General Chapter <1087> for more information. Experimental data suggests that the topiramate dissolution rate in water at 37° C. is below 0.6 mg/min/cm2.

By “sustained release” is meant predetermined continuous release of active agent to an environment over a prolonged period.

The expressions “exit,” “exit orifice,” “delivery orifice” or “drug delivery orifice,” and other similar expressions, as may be used herein include a member selected from the group consisting of a passageway, an aperture, an orifice, and a bore. The expression also includes an orifice that is formed or formable from a substance or polymer that erodes, dissolves or is leached from the outer wall to thereby form an exit orifice.

A drug “release rate” refers to the quantity of drug released from a dosage form per unit time, e.g., milligrams of drug released per hour (mg/hr). Drug release rates for drug dosage forms are typically measured as an in vitro rate of drug release, i.e., a quantity of drug released from the dosage form per unit time measured under appropriate conditions and in a suitable fluid. The dissolution tests described herein were performed on dosage forms placed in metal coil or metal cage sample holders attached to a USP Type VII bath indexer in a constant temperature water bath at 37° C. Aliquots of the release rate solutions were injected into a chromatographic system to quantify the amounts of drug released during the testing intervals.

By “release rate assay” is meant a standardized assay for the determination of the release rate of a compound from the dosage form tested using a USP Type VII interval release apparatus. It is understood that reagents of equivalent grade may be substituted in the assay in accordance with generally accepted procedures.

As used herein, unless otherwise specified, a drug release rate obtained at a specified time “following administration” refers to the in vitro drug release rate obtained at the specified time following implementation of an appropriate dissolution test. The time at which a specified percentage of the drug within a dosage form has been released may be referenced as the “Tₓ” value, where “x” is the percent of drug that has been released. For example, a commonly used reference measurement for evaluating drug release from dosage forms is the time at which 70% of drug within the dosage form has been released. This measurement is referred to as the “T₇₀” for the dosage form.

An “immediate-release dosage form” refers to a dosage form that releases drug substantially completely within a short time period following administration, i.e., generally within a few minutes to about 1 hour.

By “controlled release dosage form” is meant a dosage form that releases drug substantially continuously for many hours. Controlled release dosage forms in accord with the present invention exhibit T₇₀ values of at least about 8 to 20 hours and preferably 15 to 18 hours and more preferably about 17 hours or more. The dosage forms continuously release drug for sustained periods of at least about 8 hours, preferably 12 hours or more and, more preferably, 16-20 hours or more.

Dosage forms in accord with the present invention exhibit controlled release rates of a therapeutic agent for a prolonged period of time.

By “uniform release rate” is meant an average hourly release rate from the core that varies positively or negatively by no more than about 30% and preferably no more than about 25% and most preferably no more than 10% from either the preceding or the subsequent average hourly release rate as determined in a USP Type VII Interval Release Apparatus where the cumulative release is between about 25% to about 75%.

By “stepwise” or “ascending” rate of release is meant a first rate of release for a first period of time followed by a second rate of release for a second period of time. The first rate of release being less than the second rate of release with each rate of release being substantially uniform over its period of time of delivery. That first period of time and the second period of time can be of equal or different periods as desired.

By “prolonged period of time” is meant a continuous period of time of at least about 4 hours, preferably 6-8 hours or more and, more preferably, 10 hours or more to 24 hours or more. For example, the exemplary osmotic dosage forms described herein generally begin releasing therapeutic agent at a uniform release rate within about 2 to about 6 hours following administration and the uniform rate of release, as defined above, continues for a prolonged period of time from about 25% to until at least about 75% and preferably at least about 85% of the drug is released from the dosage form. Release of therapeutic agent continues thereafter for several more hours although the rate of release is generally slowed somewhat from the uniform release rate.

By “C” is meant the concentration of drug in the blood plasma of a subject, generally expressed as mass per unit volume, typically nanograms per milliliter. For convenience, this concentration may be referred to as “plasma drug concentration” or “plasma concentration” herein which is intended to be inclusive of drug concentration measured in any appropriate body fluid or tissue. The plasma drug concentration at any time following drug administration is referenced as Cₜᵢₘₑ, as in C₉ₕ or C₂₄ₕ, etc.

By “steady state” is meant the condition in which the amount of drug present in the blood plasma of a subject does not vary significantly over a prolonged period of time. A pattern of drug accumulation following continuous administration of a constant dose and dosage form at constant dosing intervals eventually achieves a “steady-state” where the plasma concentration peaks and plasma concentration troughs are essentially identical within each dosing interval. As used herein, the steady-state maximal (peak) plasma drug concentration is referenced as Cₘₐₓ and the minimal (trough) plasma drug concentration is referenced as Cₘᵢₙ. The times following drug administrations at which the steady-state peak plasma and trough drug concentrations occur are referenced as the Tₘₐₓ and the Tₘᵢₙ, respectively.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form dissolution rates and in vivo plasma drug concentrations.

By “high dosage” is meant drug loading therapeutic agent within the dosage form that comprises 30% or more, and preferably 40% or more, by weight of the dosage form. More particularly, the present invention provides optimal functionality when greater than about 50% of the drug layer composition is topiramate.

By “dry state” or “substantially dry state” is meant that the composition forming the drug layer of the dosage form is expelled from the dosage form in a plug-like state, the composition being sufficiently dry or so highly viscous that it does not readily flow as a liquid stream from the dosage form under the pressure exerted by the push layer.

When membrane coated osmotic drug compositions are tested for water content at various release intervals, the drug composition of a conventional osmotic dosage form releasing a solution or suspension exhibits a water content less than 1% before release, 9% water content at two hours of release and 31% water content at eight hours of release. The drug composition of the present invention exhibits a water content of less than 1% before release, 7% water content at two hours of release and 19% water content at eight hours of release, indicating a dry or substantially dry drug layer during release.

The sustained release dosage forms incorporating drug core compositions of high doses of therapeutic agent topiramate exhibiting T₇₀ values of about 10 to 20 hours and preferably 15 to 18 hours and more preferably at about 17 hours or more which release at a uniform release rate for a prolonged period of time can be prepared. Administration of such dosage forms once daily can provide therapeutically effective average steady-state plasma concentrations.

The exemplary sustained release dosage forms incorporating the drug core composition of the present invention, methods of preparing such dosage forms and methods of using such dosage forms described herein are directed to osmotic dosage forms for oral administration. In addition to osmotic systems as described herein, however, there are many other approaches to achieving sustained release of drugs from oral dosage forms known in the art. These different approaches may include, for example, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems as described in Remington's Pharmaceutical Sciences, 18^{th} Ed., pp. 1682-1685, 1990. Therapeutic agent dosage forms that operate in accord with these other approaches are encompassed by the scope of the claims below to the extent that the drug release characteristics as recited in the claims describe those dosage forms either literally or equivalently.

Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. A significant advantage of osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated in its entirety by reference herein. In particular, the following U.S. Patents, owned by the assignee of the present application, ALZA Corporation, directed to osmotic dosage forms, are each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

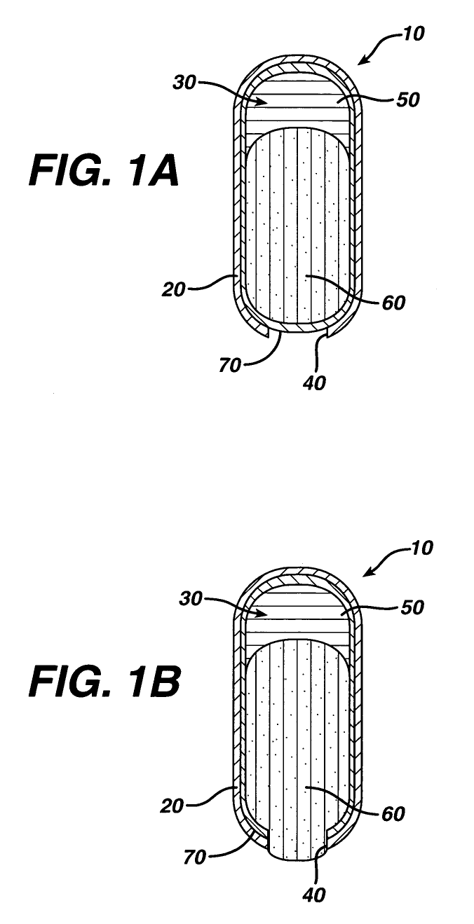

FIG. 1A and FIG. 1B illustrate an embodiment of a dosage form of this invention. Dosage form 10 comprises a wall 20 defining a compartment 30. Wall 20 is provided with an exit orifice 40. Within compartment 30, and remote from the exit orifice 40, is a push layer 50. A drug layer 60 is located within compartment 30 adjacent exit orifice 40. An optional secondary wall 70, a lubricating subcoat, may extend between drug layer 60 and the inner surface of wall 20. Secondary wall 70 may also extend between both drug layer 60 and push layer 50 and the inner surface of wall 20.

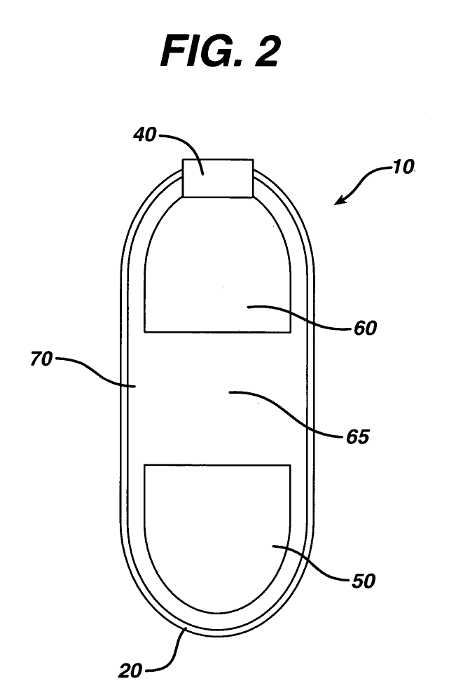

FIG. 2 illustrates the preferred embodiment of the present invention wherein dosage form 10 of FIG. 1A contains a second drug layer 65 interposed between the first drug layer 60 and push layer 30. Second drug layer 65 may be identical in all respect to first drug layer 60 except that it will have a higher concentration of active agent 61.

Drug layer 60 and drug layer 65 comprise a composition formed of a drug 61, an active agent, a carrier 62, such as a hydrophilic polymer, and optionally a disintegrant 63.

Second drug layer 65 may also be incorporated into dosage form 10. Drug layer 65 may vary from drug layer 60 to include a different drug, different carrier or different disintegrant as well as different amounts any of the same.

The drug composition layers 60 are dry or substantially dry which is intended to mean that the composition in the dosage form prior to administration has a maximum weight percent of water of less than 1%. After administration to a subject the drug composition with the dosage form remains dry or substantially dry with the maximum weight percent of water 8 hours after administration being less than 19% and is preferably between 7 wt % and 19 wt %. This would relate to the water content of the drug composition actually released from the dosage form.

More particularly, when membrane coated drug compositions are tested for water content at various release intervals, the drug composition of a conventional osmotic formulation exhibits a water content less than 1% before release, 9% water content at two hours of release and 31% water content at eight hours of release. This allows the drug composition to be released as a solution or suspension from a small exit orifice in the dosage form. The drug composition of this present invention exhibits a water content of less than 1% before release, 7% water content at two hours of release and 19% water content at eight hours of release, indicating a substantially dry drug layer during release.

The active agent drug 61 in the drug composition layer 60 is capable of drug loading between 10 mg and 450 mg and optimally of between 100 mg to 250 mg and most preferably between 160 mg and 250 mg in the composition, which unexpectedly comprises about 4% to about 60% of the drug composition and 1% to 40% of the total dosage form by weight. More preferably the active agent comprises from about 6% to about 60% of the drug composition and 2% to 36% of the total dosage form by weight.

The therapeutic agent may be provided in the drug layer in amounts from 1 μg to 750 mg per dosage form, preferably 1 mg to 500 mg per dosage form, and more preferably 100 mg to 250 mg depending upon required dosing level that must be maintained over the delivery period, i.e., the time between consecutive administrations of the dosage forms. More typically, loading of compound in the dosage forms will provide doses of compound to the subject ranging from 20 mg to 350 mg and more usually 40 mg to 200 mg per day. Generally, if a total drug dose of more than 200 mg per day is required, multiple units of the dosage form may be necessarily administered at the same time to provide the required amount of drug.

Therapeutic salts of the active agent may be represented by a member selected from the group consisting of the following: anion salts such as acetate, adipate, benzenesulfonate, benzoate, bicarbonate, bitartrate, bromide, calcium edetate, camsylate, carbonate, chloride, citrate, dihydrochloride, edetate, edisylate, estolate, fumerate, gluceptate, gluconate, glutamate, glycollylarsanilate, hexylreorinate, hydrabamine, hydrobromide, hydrochloride, hydroxynaphthoate, iodide, isethionate, lactate, lactobionate, malate, maleate, mandelate, mesylate, methylbromide, methylnitrate, mucate, napsylate, nitrate, pamoate, pantothenate, phosphate, diphosphate, polygalacturonate, salicylate, stearate, subacetate, succinate, sulfate, tannate, tartrate, teoclate, triethiodide, or cation salts such as benzathine, chloroprocaine, choline, diethanolamine, ethylenediamine, meglumine, procaine, aluminium, calcium, lithium, magnesium, potassium, sodium, zinc, polymer/drug complexes such as cyclodextrinates, polyvinylpyrrolidonates, and the like.

For most applications, dosage forms having 100-500 mg of drug per dosage form are convenient. While preparations described herein may include 600-1200 mg of drug, the dosage forms containing lesser amounts of drug may be multiply dosed at the same time to obtain similar delivery results as with dosage forms having higher drug loading.

When drug 61 is present in high dosage amounts, greater than 30% of the dosage form by weight, and/or greater than about 54% of the drug layer composition by weight, the present invention provides a beneficial increase in dissolution of the drug.

When the active agent drug 61 comprises topiramate or one of its pharmaceutically acceptable salts, the doses of lowly soluble topiramate that can be incorporated into the dosage form of the present invention can range from about 10 milligrams to about 750 milligrams, with an especially preferred range of 100 mg to 300 mg depending upon the required dosing level that must be maintained over the delivery period, i.e., the time between consecutive administrations of the dosage forms. More typically, loading of compound in the dosage forms will provide doses of compound to the subject ranging from 10-600 mg per day, more usually 100 mg to 400 mg per day. For the present invention optimum performance has been demonstrated with drug loading of about 100 mg to about 450 mg and more preferably 160 mg to 340 mg, comprising up to 80% or 90% of the drug composition by weight. The drug layer typically will be a dry or substantially dry composition formed by compression of the carrier and drug composition as one layer and the expandable or push layer as the second layer. The expandable layer will push the drug layer from the exit orifice as the push layer imbibes fluid from the environment of use, and the exposed drug layer will be eroded to release the drug into the environment of use.

Topiramate is in the therapeutic category of anti-convulsants although the drug may be therapeutic for other indications as well.

Topiramate exhibits low solubility of about 9.8 mg/ml to 13.0 mg/ml with solubility of neat topiramate measured in de-ionized water being 12 mg/ml.

Topiramate therapy is recommended at dosing initially at 25-50 mg/day followed by titration in weekly increments of 25-50 mg upward to an effective dose. Typical effective dose can be up to 400 mg per day.

Topiramate immediate release is typically administered at a starting dose of 100 mg/day, administered in two divided doses (BID). The effective dose range has been determined to be generally 200 mg/day to 400 mg/day. Observation of tolerability and need for additional clinical effect over the starting dose often results in the dose being increased in increments of 100 mg/day to 200 mg/day, on a BID schedule, at intervals of no less than one week. Several weeks of treatment often are required to obtain the full therapeutic response. Concurrently with observation, plasma concentrations in a subject may be determined by clinical assay to determine a correlation between tolerability and clinical effect and blood plasma concentrations of drug. Plasma concentrations may range from 5 to 5000 ng/ml (nanograms per milliliter), more typically 25 to 2500 ng/ml, of compound.

Comparable standards of observation of tolerability and clinical effect and clinical assays for blood plasma concentration that have been employed with immediate release dosage forms of the compounds may be employed to adjust the daily dose of the active agent in the sustained release dosage forms of this invention that are most appropriate for a particular subject. Generally, the lowest dose of compound providing the desired clinical effect will be utilized. Such dosages may be in the range of 10 mg/day to 1200 mg/day, more often in the range of 50 mg/day to 800 mg/day, and most often in the range of 100 mg/day to 600 mg/day, delivered to the subject over a prolonged period of time. Preferably the dose will be selected to provide a daily dose in the range of 50 mg/day to 800 mg/day, and most preferably from 100 mg/day to 600 mg/day.

Dosage forms of the present invention which provide a stepwise release rate of the active compound may in appropriate circumstances allow a lesser amount of compound per dosage form per day than would be calculated from simply multiplying the dose of active agent in the immediate release product by the number of times it is recommended to administer the immediate release product in a day. In other circumstances, an equal or greater daily dosage of the active agent may be required to elicit a desired patient response.

Even at high dosage levels in which the active compound is present from 40% to 90% by weight of the drug layer compositions, the instant dosage forms and devices are able to effectively release the required amount of active compound over a prolonged period of time at a stepwise release rate. Preferably, the weight percent of active compound in a drug layer compositions of the invention will be 75% or less, and most preferably less than 70%, but greater than 50%, most preferably greater than 65%, based on the weight of drug layer composition, to allow for dosage forms that may be easily swallowed. In circumstances where it is desirable to administer an amount of drug that would exceed 75% of the drug layer compositions, it is usually preferred to simultaneously administer two tablets or more of the dosage form with a total drug loading equal to the greater amount that would have been used in the single tablet.

It has been found convenient for topiramate, for example, to prepare once-a-day dosage forms in accordance with this invention having 100 mg, 160 mg, 200 mg, 250 mg, and 450 mg of topiramate per dosage form. After an initial start-up period, usually approximately 2-3 hours or less, the dosage forms provide a stepwise rate of release of compound over a prolonged period of time, typically 4 hours to 20 hours or more, often for 4 hours to 16 hours, and more usually at a uniform rate for a time period of 4 hours to 10 hours. At the end of a prolonged period of stepwise release, the rate of release of drug from the dosage form may decline somewhat over a period of time, such as several hours. The dosage forms provide therapeutically effective amounts of drug for a broad range of applications and individual subject needs.

Upon initial administration, the dosage forms may provide a drug concentration in the plasma of the subject that increases over an initial period of time, typically several hours or less, and then provide a relatively constant concentration of drug in the plasma over a prolonged period of time, typically 4 hours to 24 hours or more. The release profiles of the dosage forms of this invention provide release of drug over the entire 24-hour period corresponding to once-a-day administration, such that steady state concentration of drug in blood plasma of a subject may be maintained at therapeutically effective levels over a 24 hour period after administration of the sustained release dosage form. Steady state plasma levels of drug may typically be achieved after twenty-four hours or, in some cases, several days, e.g., 2-6 days, in most subjects.

Structural polymer carrier 62 comprises a hydrophilic polymer which provides cohesiveness to the blend so durable tablets can be made.

The hydrophilic polymer provides a hydrophilic polymer particle in the drug composition that contributes to the uniform release rate of active agent and controlled delivery pattern. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000, number-average molecular weight, represented by poly(alkali carboxymethylcellulose), poly(sodium carboxymethylcellulose), poly(potassium carboxymethylcellulose) and poly(lithium carboxymethylcellulose). The drug composition can comprise a hydroxypropylalkylcellulose of 9,200 to 125,000 number-average molecular weight for enhancing the delivery properties of the dosage form as represented by hydroxypropylethylcellulose, hydroxypropyl methylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among those polymers are the poly(ethylene oxide) of 100,000-300,000 number average molecular weight. Carriers that erode in the gastric environment, i.e., bioerodible carriers, are especially preferred.

The hydrophilic polymer carrier 62 is also in a reduced amount comprising from about 10% to 86% of the drug composition and 6% to 52% of the total dosage form by weight. More preferably the hydrophilic polymer carrier comprises from about 30% to 86% of the drug composition and 18% to 22% of the total dosage form by weight.

Carrier 62 provides a hydrophilic polymer particle in the drug composition that contributes to the controlled delivery of active agent. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 1,000,000 400,000 number-average molecular weight, represented by poly(alkali carboxymethylcellulose), poly(sodium carboxymethylcellulose), poly(potassium carboxymethylcellulose) poly(calcium carboxymethylcellulose), and poly(lithium carboxymethylcellulose). The drug composition can comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among those polymers are the poly(ethylene oxide) of 100,000-300,000 number average molecular weight. Carriers that erode in the gastric environment, i.e., bioerodible carriers, are especially preferred.

Other carriers that may be incorporated into drug layer 60 include carbohydrates that exhibit sufficient osmotic activity to be used alone or with other osmoagents. Such carbohydrates comprise monosaccharides, disaccharides and polysaccharides. Representative examples include maltodextrins (i.e., glucose polymers produced by the hydrolysis of grain starch such as rice or corn starch) and the sugars comprising lactose, glucose, raffinose, sucrose, mannitol, sorbitol, xylitol, cyclodextrin and the like. Preferred maltodextrins are those having a dextrose equivalence (DE) of 20 or less, preferably with a DE ranging from about 4 to about 20, and often 9-20. Maltodextrin having a DE of 9-12 and molecular weight of about 1,600 to 2,500 has been found most useful.

Carbohydrates described above, preferably the maltodextrins, may be used in the drug layer 60 without the addition of an osmoagent, and obtain the desired release of therapeutic agent from the dosage form, while providing a therapeutic effect over a prolonged period of time and up to 24 hours with once-a-day dosing.

The presently preferred range of concentration of structural polymer within the present invention for osmotic delivery systems is 6 to 52 weight percent of polyoxyethylene 100,000 to 200,000 molecular weight (Polyox N80), with an especially preferred range of 18 to 52 weight percent.

A disintegrant 63 may be utilized in the drug layer composition as well. Exemplary of the disintegrants are starches, clays, celluloses, algins and gums and crosslinked starches, celluloses and polymers. Representative disintegrants include corn starch, potato starch, croscarmellose, crospovidone, sodium starch glycolate, Veegum HV, methylcellulose, agar, bentonite, carboxymethylcellulose, alginic acid, guar gum and the like.

The disintegrant is in an amount comprising from about 1% to about 20% of the drug composition and preferably in an amount comprising from about 3% to 8% of the drug composition and 1% to 5% of the total dosage form by weight. More preferably the disintegrant comprises from about 4% to about 6% of the drug composition and 2% to 4% of the total dosage form by weight.

The present invention releases the active agent at a controlled rate over a prolonged period of time providing from a high drug loading dosage form and is capable of maintaining bioavailability equivalent to dosage forms with lower drug loading. The present invention uses no surfactants and operates on a dispersion mechanism rather than a solubility enhancement mechanism to achieve between about 75% and about 98% bioavailability and preferably about 96% bioavailability similar to conventional osmotic delivery systems handling lower doses of active agent.

Drug layer 60 is optimally manufactured as a mixture from particles by comminution that produces the size of the drug and the size of the accompanying polymer used in the fabrication of the drug layer, typically as a core containing the composition, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy-grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Remington's Pharmaceutical Sciences, 18th Ed., pp. 1615-1632 (1990); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Push layer 50 is an expandable layer comprising a push-displacement composition in contacting layered arrangement with the drug layer 60. It comprises a polymer that imbibes an aqueous or biological fluid and swells to push the drug composition through the exit means of the device. Representatives of fluid-imbibing displacement polymers comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

The osmagent, also known as osmotic solute and osmotically effective agent, which exhibits an osmotic pressure gradient across the outer wall and subcoat, comprises a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid raffinose, sucrose, glucose, lactose, sorbitol, inorganic salts, organic salts and carbohydrates.

Exemplary solvents suitable for manufacturing the hydroactivated layer and the wall comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Wall 20 is formed to be permeable to the passage of an external fluid, such as water and biological fluids, and it is substantially impermeable to the passage of active agent, osmagent, osmopolymer and the like. As such, it is semipermeable. The selectively semipermeable compositions used for forming the wall are essentially nonerodible and they are insoluble in biological fluids during the life of the dosage form.

Representative polymers for forming wall 20 comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions forming wall 20 typically include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Additional semipermeable polymers for forming outer wall 20 comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltrimethylammonium chloride); and semipermeable polymers exhibiting a fluid permeability of 10-5 to 10-2 (cc. mil/cm hr.atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio.

Wall 20 also can comprise a flux-regulating agent. The flux regulating agent is a compound added to assist in regulating the fluid permeability or flux through wall 20. The flux-regulating agent can be a flux enhancing agent or a decreasing agent. The agent can be preselected to increase or decrease the liquid flux. Agents that produce a marked increase in permeability to fluid such as water are often essentially hydrophilic, while those that produce a marked decrease to fluids such as water, are essentially hydrophobic. The amount of regulator in the wall when incorporated therein generally is from about 0.01% to 30% by weight or more. The flux regulator agents in one embodiment that increase flux include polyhydric alcohols, polyalkylene glycols, poilyalkylenediols, polyesters of alkylene glycols, and the like. Typical flux enhancers include polyethylene glycol 300, 400, 600, 1500, 4000, 6000 and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol 1,2,4-hexanetriol, 1,3,6-hexanetriol and the like; esters such as ethylene glycol dipropionate, ethylene glycol butyrate, butylene glycol dipropionate, glycerol acetate esters, and the like. Representative flux decreasing agents include phthalates substituted with an alkyl or alkoxy or with both an alkyl and alkoxy group such as diethyl phthalate, dimethoxyethyl phthalate, dimethyl phthalate, and [di(2-phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form the wall 20 for imparting flexibility and elongation properties to the wall, for making wall 20 less-to-nonbrittle and to render tear strength, include phthalate plasticizers such as dibenzyl phthalate, dihexyl phthalate, butyl octyl phthalate, straight chain phthalates of six to eleven carbons, di-isononyl phthalate, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The dosage form may comprise a device comprising (1) a semipermeable wall that forms a compartment; (2) at least a first drug composition layer in the compartment; (3) an exit orifice in the semipermeable wall; and optionally, (4) a secondary wall between at least the drug composition layers and the semipermeable wall that reduces friction between the external surface of the drug layer compositions and the inner surface of wall 20, promotes release of the drug composition from the compartment and reduces the amount of drug composition remaining in the compartment at the end of the delivery period.

The optional secondary wall 70 is in contacting position with the inner surface of the semipermeable wall 20 and at least the external surface of the drug layer; although the secondary wall 70 may extend to and contact the external surface of the push layer. Optional secondary wall 70 may be formed as a coating applied over the compressed core comprising the drug layers and the push layer. The outer semipermeable wall 20 surrounds and encases the inner, secondary wall 70. Secondary wall 70 is preferably formed as a subcoat of at least the surface of drug layer 60 and optional drug layer 65, and optionally the entire external surface of the compacted drug layer 60 and drug layer 65, and the push layer 50. When the semipermeable wall 20 is formed as a coat of the composite formed from the drug layer 60, the push layer 50 and the secondary wall 70, contact of the semipermeable wall 20 with the inner coat is assured.

Secondary wall 70 facilitates release of drug from the dosage forms of the invention. In dosage forms in which there is high drug loading, i.e., 40% or greater active agent in the drug layer based on the overall weight of the drug layer, and no secondary wall, it has been observed that significant residual amounts of drug may remain in the device after the period of delivery has been completed. In some instances, amounts of 20% or greater may remain in the dosage form at the end of a twenty-four hour period when tested in a release rate assay.

The amount of residual drug may be reduced by the addition of secondary wall 70 formed as an inner coat of a flow-promoting agent, i.e., an agent that lowers the frictional force between the outer, semi-permeable membrane wall 20 and the external surface of the drug composition layers. The secondary wall or inner coat 70 apparently reduces the frictional forces between the semipermeable wall 20 and the outer surface of the drug layer, thus allowing for more complete delivery of drug from the device. Particularly in the case of active compounds having a high cost, such an improvement presents substantial economic advantages since it is not necessary to load the drug layer with an excess of drug to insure that the minimal amount of drug required will be delivered.

Secondary wall 70 typically may be 0.01 to 5 mm thick, more typically 0.5 to 5 mm thick, and it comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, hydroxyalkyl alkylcelluloses, e.g., hydroxypropyl methylcellulose, povidone[poly(vinylpyrrolidine)], polyethylene glycol and mixtures thereof. The hydroxyalkylcelluloses comprise polymers having a 9,500 to 1,250,000 number-average molecular weight. For example, hydroxypropyl celluloses having number average molecular weights of between 80,000 and 850,000 are useful. The flow-promoting layer may be prepared from conventional solutions or suspensions of the aforementioned materials in aqueous solvents or inert organic solvents. Preferred materials for the subcoat or flow promoting layer include hydroxypropyl cellulose, hydroxyethyl cellulose, hydroxypropyl methyl cellulose, povidone [poly(vinylpyrrolidone)], polyethylene glycol, and mixtures thereof. More prefered are mixtures of hydroxypropyl cellulose and povidone, prepared in organic solvents, particularly organic polar solvents such as lower alkanols having 1-8 carbon atoms, preferably ethanol, mixtures of hydroxyethyl cellulose and hydroxypropyl methyl cellulose prepared in aqueous solution, and mixtures of hydroxyetyyl cellulose and polyethylene glycol prepared in aqueous solution. Most preferably, the subcoat consists of a mixture of hydroxypropyl cellulose and povidone prepared in ethanol. Conveniently, the weight of the subcoat applied to the bilayer core may be correlated with the thickness of the subcoat and residual drug remaining in a dosage form in a release rate assay such as described herein. During manufacturing operations, the thickness of the subcoat may be controlled by controlling the weight of the subcoat taken up in the coating operation.

When the secondary wall 70 is formed as a subcoat, i.e., by coating onto the tabletted bilayer composite drug layer and push layer, the subcoat can fill in surface irregularities formed on the bilayer core by the tabletting process. The resulting smooth external surface facilitates slippage between the coated bilayer composite and the semipermeable wall during dispensing of the drug, resulting in a lower amount of residual drug composition remaining in the device at the end of the dosing period. When wall 7 is fabricated of a gel-forming material, contact with water in the environment of use facilitates formation of the gel or gel-like inner coat having a viscosity that may promote and enhance slippage between outer wall 2 and drug layer 60.

Pan coating may be conveniently used to provide the completed dosage form, except for the exit orifice. In the pan coating system, the subcoat on the wall-forming compositions is deposited by successive spraying of the respective composition on the bilayered core comprising the drug layer and the push layer accompanied by tumbling in a rotating pan. A pan coater is used because of its availability at commercial scale. Other techniques can be used for coating the drug core. Finally, the wall or coated dosage form are dried in a forced-air oven, or in a temperature and humidity controlled oven to free the dosage form of solvent. Drying conditions will be conventionally chosen on the basis of available equipment, ambient conditions, solvents, coatings, coating thickness, and the like.

Other coating techniques can also be employed. For example, the semipermeable wall and the subcoat of the dosage form can be formed in one technique using the air-suspension procedure. This procedure consists of suspending and tumbling the bilayer core in a current of air, an inner subcoat composition and an outer semipermeable wall forming composition, until, in either operation, the subcoat and the outer wall coat is applied to the bilayer core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent. An Aeromatic® air-suspension coater can be used employing a cosolvent.

The dosage form of the invention may be manufactured by standard techniques. For example, the dosage form may be manufactured by the wet granulation technique. In the wet granulation technique, the drug and the ingredients comprising the first layer or drug composition are blended using an organic solvent, such as denatured anhydrous ethanol, as the granulation fluid. The ingredients forming the first layer or drug composition are individually passed through a preselected screen and then thoroughly blended in a mixer. Next, other ingredients comprising the first layer can be dissolved in a portion of the granulation fluid, such as the solvent described above. Then, the latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate is added to the drug granulation, then put into milling jars and mixed on a jar mill for 10 minutes. The composition is pressed into a layer, for example, in a Manesty® press or a Korsch LCT press. The speed of the press is set at 15 rpm and the maximum load set at 4 tons. The first layer is pressed against the composition forming the second layer and the bilayer tablets are fed to a dry coater press, e.g., Kilian® Dry Coater press, and surrounded with the drug-free coat, followed by the exterior wall solvent coating.

In another manufacture the beneficial drug and other ingredients comprising the first layer facing the exit means are blended and pressed into a solid layer. The layer possesses dimensions that correspond to the internal dimensions of the area the layer is to occupy in the dosage form, and it also possesses dimensions corresponding to the second layer for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, a layer of osmopolymer composition is placed in contact with the layer of drug in a like manner. The layering of the drug formulation and the osmopolymer layer can be fabricated by conventional two-layer press techniques. The two contacted layers are first coated with a subcoat and an outer semipermeable wall. The air-suspension and air-tumbling procedures comprise in suspending and tumbling the pressed, contacting first and second layers in a current of air containing the delayed-forming composition until the first and second layers are surrounded by the wall composition.

Another manufacturing process that can be used for providing the compartment-forming composition comprises blending the powdered ingredients in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

The dosage form of the invention is provided with at least one exit orifice. The exit orifice cooperates with the drug core for the uniform release of drug from the dosage form. The exit orifice can be provided during the manufacture of the dosage form or during drug delivery by the dosage form in a fluid environment of use. The expression “exit orifice” as used for the purpose of this invention includes a member selected from the group consisting of a passageway; an aperture; an orifice; and a bore. The expression also includes an orifice that is formed from a substance or polymer that erodes, dissolves or is leached from the outer coat or wall or inner coat to form an exit orifice. The substance or polymer may include an erodible poly(glycolic) acid or poly(lactic) acid in the outer or inner coats; a gelatinous filament; a water-removable poly(vinyl alcohol); a leachable compound, such as a fluid removable pore-former selected from the group consisting of inorganic and organic salt, oxide and carbohydrate. An exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice. The exit orifice can have any shape, such as round, triangular, square, elliptical and the like for the uniform metered dose release of a drug from the dosage form. The dosage form can be constructed with one or more exits in spaced apart relation or one or more surfaces of the dosage form. The exit orifice can be preformed by drilling, including mechanical and laser drilling, through the outer coat, the inner coat, or both. Exits and equipment for forming exits are disclosed in U.S. Pat. Nos. 3,845,770 and 3,916,899, by Theeuwes and Higuchi; in U.S. Pat. No. 4,063,064, by Saunders, et al.; and in U.S. Pat. No. 4,088,864, by Theeuwes, et al.

With respect to the 100-450 mg dosage forms prepared as described herein, it has been found that, for a 100 mg dosage form having a core diameter of about 3/16 inch, an exit orifice of 95-180 mils, preferably 140-150 mils, and most preferably 145 mils, provides an effective release profile. For a 200 mg dosage form having a core diameter of about ¼ inch, an exit orifice of 190-210 mils, preferably 195-205 mils, and most preferably 200 mils, provides an effective release profile. For a 300 mg dosage form having a core diameter of about 9/32 inch, an exit orifice of 215-235 mils, preferably 220-230 mils, and most preferably 225 mils, provides an effective release profile. For a 400 mg dosage form having a core diameter of about 5/16 inch, an exit orifice of 240-260 mils, preferably 245-255 mils, and most preferably 250 mils, provides an effective release profile. The dosage forms release drug at a rate that varies less than 30% from the mean rate of release measured over a prolonged period of time. Preferably, the devices release drug at a stepwise rate of release over a prolonged period of time.

Dosage forms of the present invention release drug at a stepwise increasing rate of release over a prolonged period of time as determined in a standard release rate assay such as that described herein. When administered to a subject, the dosage forms of the invention provide blood plasma levels of drug in the subject that are less variable over a prolonged period of time than those obtained with immediate release dosage forms. When the dosage forms of this invention are administered on a regular, once-a-day basis, the dosage forms of the invention provide steady state plasma levels of drug such that the difference between Cₘₐₓ and Cₘᵢₙ over the 24-hour period is substantially reduced over that obtained from administration of an immediate release product that is intended to release the same amount of drug in the 24-hour period as is provided from the dosage forms of the invention.

Measurements of release rate are typically made in vitro, in acidified water to provide a simulation of conditions in gastric fluid, and are made over finite, incremental time periods to provide an approximation of instantaneous release rate. Information of such in vitro release rates with respect to a particular dosage form may be used to assist in selection of dosage form that will provide desired in vivo results. Such results may be determined by present methods, such as blood plasma assays and clinical observation, utilized by practitioners for prescribing available immediate release dosage forms.

Dosage forms of this invention may provide blood plasma concentrations in the range of 5 to 5000 ng/ml, more typically in the range of 25 to 1200 ng/ml. Blood plasma of a subject to whom the dosage form has been administered may be assayed to determine the concentration of active agent in blood plasma as a function of time after the dosage form has been administered. This in effect allows for titration of the amount of drug to be administered to a subject over time.

It has been found that dosage forms of the present invention having release rate profiles as defined herein will provide to a patient a substantially ascending steady state blood plasma concentration and a sustained therapeutic effect of active agent, after administration of the dosage form, over a prolonged period of time. The sustained release dosage forms of this invention may demonstrate less variability in drug plasma concentration over a 24-hour period than do immediate release formulations, which characteristically create significant peaks in drug concentration shortly or soon after administration to the subject.

At steady state, the difference between Cₘₐₓ and Cₘᵢₙ of drug in plasma of the subject to which the dosage form is administered over a 24-hour period after administration of a once-a-day dosage form is less than the difference between Cₘₐₓ and Cₘᵢₙ for an immediate release dosage form(s) that is administered to provide the same total amount of drug over the period. While some subject-to-subject variability will be expected, the quotient formed from [Cₘₐₓ−Cₘᵢₙ]/C_{avg} for a once-a-day dosage form may be on the order of 3 or less, often 2 or less, preferably 1 or less and most preferably ½ or less. For example, if at steady state Cₘₐₓ is 200 ng/ml and Cₘᵢₙ is 100 ng/ml, the quotient will be less than 1. If Cₘₐₓ is 200 and Cₘᵢₙ is 150, the quotient will be less than ⅓. If Cₘₐₓ is 100 ng/ml and Cₘᵢₙ is 25 ng/ml, then the quotient is less than 3. Generally, the quotient determined from observed plasma concentrations can be expected to be larger with dosage forms containing lesser amounts of drug, although absolute variations in concentration may be smaller.

The practice of the foregoing methods by orally administering a dosage form of the invention to a subject once a day for therapeutic treatment is preferred. A preferred method of manufacturing dosage forms of the present invention is generally described below. All percentages are weight percent unless otherwise noted.

### EXAMPLE 1

Topiramate Capsule Shaped Bilayer 100 mg System for Uniform Delivery

A dosage form adapted, designed and shaped as an osmotic drug delivery device is manufactured as follows as illustrated in FIG. 1A.

Preparation of the Drug Layer Granulation

60.0 g of topiramate, 25.45 g of polyethylene oxide with average molecular weight of 200,000, 5.0 g of cross-linked povidone with average molecular weight of more than 1,000,000(PVP XL or PVP XL-10) and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 1.0 g of magnesium stearate.

Preparation of the Osmotic Push Layer Granulation

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000 molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Bilayer Core Compression

Next, the topiramate drug composition and the push composition are compressed into bilayer tablets on the Korsch Tablet Press. The press is set at 15 RPM. First, 167 mg of the topiramate composition is added to the die cavity and pre-compressed, then, 111 mg of the push composition is added and the layers are pressed under a pressure head of approximately 4 tons into a 3/16″ (0.476 cm) diameter bilayer longitudinal arrangement.

Preparation of the Subcoat Solution and Subcoated System

The bilayered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the bilayered arrangements in a pan coater until approximately 20 mg of laminate is applied to each tablet.

Preparation of the Rate Controlling Membrane and Membrane Coated System

The bilayered subcoated cores are coated with a semi-permeable wall. The wall forming composition comprises 99% cellulose acetate having a 39.8% acetyl content and 1% poloxamer, or polyoxyethylene-polyoxypropylene block copolymer, comprising a 7,680-9,510 average molecular weight. The wall-forming composition is dissolved in an acetone:water (99:1 wt:wt) co solvent to make a 5% solids solution. The wall-forming composition is sprayed onto and around the bilayered arrangements in a pan coater until approximately 40 mg of membrane is applied to each tablet.

Drilling of Membrane Coated Systems

Next, a 145 mil (3.7 mm) exit passageway is drilled through the semi-permeable wall to connect the drug layer with the exterior of the dosage system.

Drying of Drilled Coated Systems

The residual solvent is removed by drying for 230 hours as 45 C. and 40% humidity.

Color and Clear Overcoats

Optional color or clear coats solutions are prepared in a covered stainless steel vessel. For the color coat 88 parts of purified water is mixed with 12 parts of Opadry II [color not critical] until the solution is homogeneous. For the clear coat 95 parts of purified water is mixed with 5 parts of Opadry Clear until the solution is homogeneous. The dried cores prepared as above are placed into a rotating, perforated pan coating unit. The coater is started and after the coating temperature is attained (35-45° C.), the color coat solution is uniformly applied to the rotating tablet bed. When sufficient amount of solution has been applied, as conveniently determined when the desired color overcoat weight gain has been achieved, the color coat process is stopped. Next, the clear coat solution is uniformly applied to the rotating tablet bed. When sufficient amount of solution has been applied, or the desired clear coat weight gain has been achieved, the clear coat process is stopped. A flow agent (e.g., Carnuba wax) is applied to the tablet bed after clear coat application.

The dosage form produced by this manufacture is designed to deliver 100 mg of topiramate in a controlled delivery pattern from the drug-containing core. The drug layer contains 60% topiramate, 25.45% polyethylene oxide possessing a 200,000 molecular weight, 6% of cross-linked povidone with average molecular weight of more than 1,000,000 (PVP XL), and 4% of polyvinylpyrrolidone (Povidone K29-32), 0.05% butylated hydroxytoluene, 0.5% of magnesium stearate and 1.0% stearic acid. The push composition is comprised of 64.3% polyethylene oxide with a 7,000,000 molecular weight, 30% sodium chloride, 5% polyvinylpyrrolidone possessing an average molecular weight of 40,000, 1% ferric oxide, 0.4% butylated hydroxytoluene, and 0.25% stearic acid. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% poloxamer. The dosage form comprises one passageway, 145 mils (3.7 mm) on the center of the drug side.

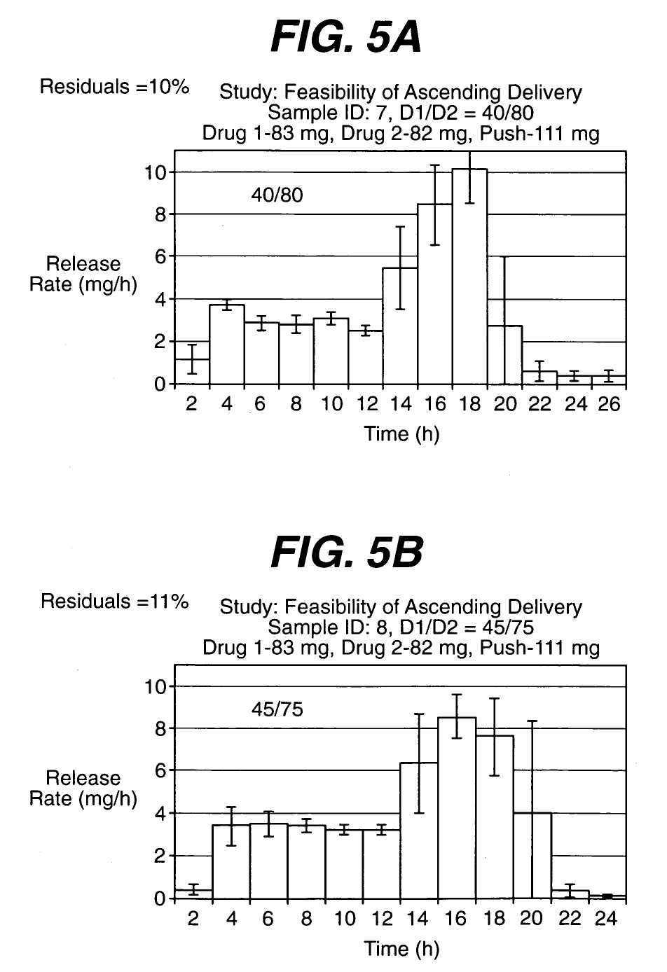

The system diagram is shown in FIG. 1A. System performance is shown in FIG. 4.

### EXAMPLE 2

### Topiramate Capsule Shaped Trilayer 100 mg System for Stepwise Delivery

A dosage form adapted, designed and shaped as an osmotic drug delivery device is manufactured as follows.

Preparation of the First Drug Layer Granulation

50.0 g of topiramate, 40.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Second Drug Layer Granulation

Second, 70.0 g of topiramate, 20.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Osmotic Push Layer Granulation

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000, molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Trilayer Core Compression

Next, the topiramate drug 1 composition, topiramate drug 2 composition and the push composition are compressed into tri-layer tablets on the Carver Tablet Press. First, 83 mg of the topiramate 1 composition is added to the die cavity and pre-compressed, then 83 mg of the topiramate 2 composition is added to the die cavity and pre-compressed, finally 111 mg of the push composition is added and the layers are pressed under a pressure head of approximately ¼ metric ton into a 3/16″ (0.476 cm) diameter tri-layer longitudinal arrangement.

Preparation of the Subcoat Solution and Subcoated System

The tri-layered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000 . The wall-forming composition is dissolved in anhydrous ethyl alcohol, to make an 8% solids solution. The wall-forming composition is sprayed onto and around the tri-layered arrangements in a pan coater until approximately 20 mg of laminate is applied to each tablet.

Preparation of the Rate Controlling Membrane and Membrane Coated System

The tri-layered subcoated cores are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the subcoated tri-layered arrangements in a pan coater until approximately 51 mg of membrane is applied to each tablet.

Drilling of Membrane Coated Systems

Next, a 145 mil (3.7 mm) exit passageway is drilled through the semi-permeable wall to connect the drug layer with the exterior of the dosage system.

Drying of Drilled Coated Systems

The residual solvent is removed by drying for 112 hours at 45° C. and ambient humidity.

The dosage form produced by this manufacture is designed to deliver 100 mg of topiramate in a stepwise delivery pattern from the drug-containing core. The first drug layer contains 50% topiramate, 40% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The second drug layer contains 70% topiramate, 20% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The push composition is comprised of 64.3% polyethylene oxide with a 7,000,000, molecular weight, 30% sodium chloride, 5% polyvinylpyrrolidone possessing an average molecular weight of 40,000, 0.4% ferric oxide, 0.05% butylated hydroxytoluene, and 0.25% stearic acid. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% polyethylene glycol. The dosage form contains one passageway, 145 mils (3.7 mm) on the center of the drug side.

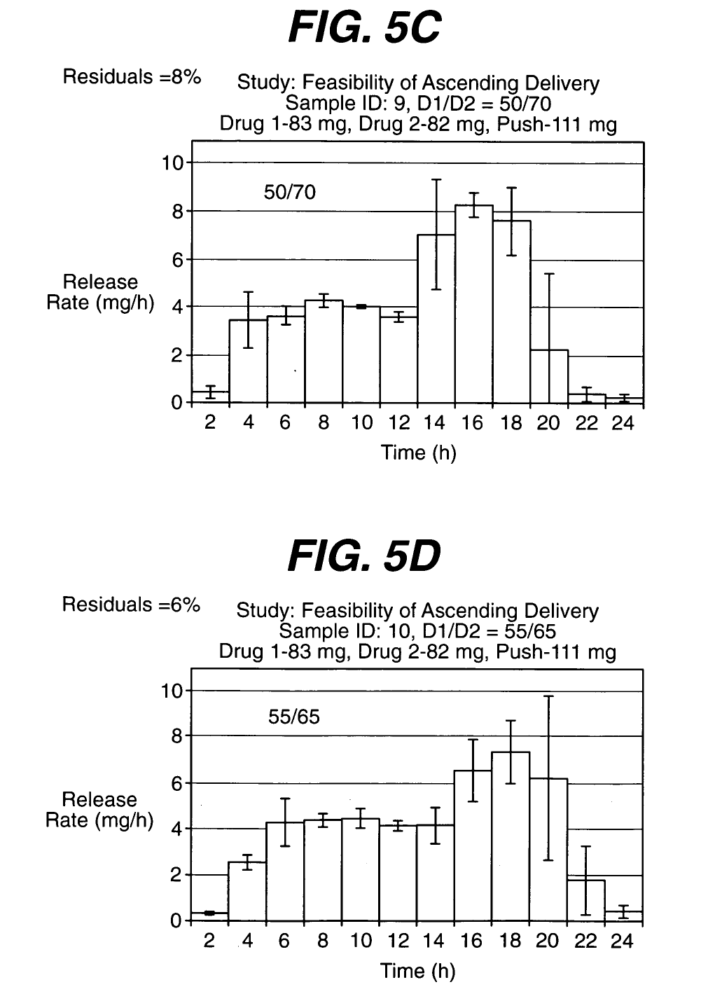

Proposed system diagram is shown in FIG. 2. Examples of system performance are shown in FIG. 5 for various drug levels including FIG. 5C representing the drug levels obtained from the dosage form prepared as in Example 2, herein.

### EXAMPLE 3

### Topiramate Capsule Shaped Trilayer 100 mg System for Stepwise Delivery

A dosage form adapted, designed and shaped as an osmotic drug delivery device is manufactured as follows as illustrated in FIG. 2.

Preparation of the First Drug Layer Granulation

55.0 g of topiramate, 35.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Second Drug Layer Granulation

Second, 65.0 g of topiramate, 25.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Osmotic Push Layer Granulation

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000, molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Trilayer Core Compression

Next, the topiramate first drug composition, topiramate second drug composition and the push composition are compressed into tri-layer tablets on the Carver Tablet Press. First, 83 mg of the topiramate first drug composition is added to the die cavity and pre-compressed, then 83 mg of the topiramate second drug composition is added to the die cavity and pre-compressed, finally 111 mg of the push composition is added and the layers are pressed under a pressure head of approximately ¼ a metric ton into a 3/16″ (0.476 cm) diameter tri-layer longitudinal arrangement.

Preparation of the Subcoat Solution and Subcoated System

The tri-layered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the tri-layered arrangements in a pan coater until approximately 20 mg of laminate is applied to each tablet.

Preparation of the Rate Controlling Membrane and membrane Coated System

The tri-layered subcoated cores are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the subcoated tri-layered arrangements in a pan coater until approximately 55 mg of membrane is applied to each tablet.

Drilling of Membrane Coated Systems

Next, a 145 mil (3.7 mm) exit passageway is drilled through the semi-permeable wall to connect the drug layer with the exterior of the dosage system. The residual solvent is removed by drying for 72 hours at 45° C. and 45% humidity.

Drying of Drilled Coated Systems

The dosage form produced by this manufacture is designed to deliver 100 mg of topiramate in a stepwise delivery pattern from the drug-containing core. The first drug layer contains 55% topiramate, 35% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The second drug layer contains 65% topiramate, 25% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The push composition is comprised 64.3% polyethylene oxide comprising a 7,000,000, molecular weight, 30% sodium chloride, 5% polyvinylpyrrolidone possessing an average molecular weight of 40,000, 0.4% ferric oxide, 0.05% butylated hydroxytoluene, and 0.25% stearic acid. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% polyethylene glycol. The dosage form contains one passageway, 145 mils (3.7 mm) on the center of the drug side.

The system diagram is shown in FIG. 2. System performance is shown in FIG. 5D.

### EXAMPLE 4

### Topiramate Capsule Shaped Trilayer 200 mg System for Stepwise Delivery

A dosage form adapted, designed and shaped as an osmotic drug delivery device is manufactured as follows.

Preparation of the First Drug Layer Granulation

50.0 g of topiramate, 40.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Second Drug Layer Granulation

Second, 70.0 g of topiramate, 20.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Osmotic Push Layer Granulation

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000, molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Trilayer Core Compression

Next, the topiramate first drug composition, topiramate second drug composition and the push composition are compressed into tri-layer tablets on the Carver Tablet Press. First, 166 mg of the topiramate first drug composition is added to the die cavity and pre-compressed, then 166 mg of the topiramate second drug composition is added to the die cavity and pre-compressed, finally 222 mg of the push composition is added and the layers are pressed under a pressure head of approximately ¼ a metric ton into a ¼″ (0.635 cm) diameter tri-layer longitudinal arrangement.

Preparation of the Subcoat Solution and Subcoated System

The tri-layered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the tri-layered arrangements in a pan coater until approximately 20 mg of laminate is applied to each tablet.

Preparation of the Rate Controlling Membrane and Membrane Coated System

The tri-layered subcoated cores are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the subcoated tri-layered arrangements in a pan coater until approximately 55 mg of membrane is applied to each tablet.

Drilling of Membrane Coated Systems

Next, a 145 mil (3.7 mm) exit passageway is drilled through the semi-permeable wall to connect the drug layer with the exterior of the dosage system. The residual solvent is removed by drying for 72 hours at 45° C. and 45% humidity.

Drying of Drilled Coated Systems

The dosage form produced by this manufacture is designed to deliver 200 mg of topiramate in a stepwise delivery pattern from the drug-containing core. The first drug layer contains 50% topiramate, 40% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The second drug layer contains 70% topiramate, 20% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The push composition is comprised 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene, and 0.25% stearic acid. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% polyethylene glycol. The dosage form contains one passageway, 145 mils (3.7 mm) on the center of the drug side.

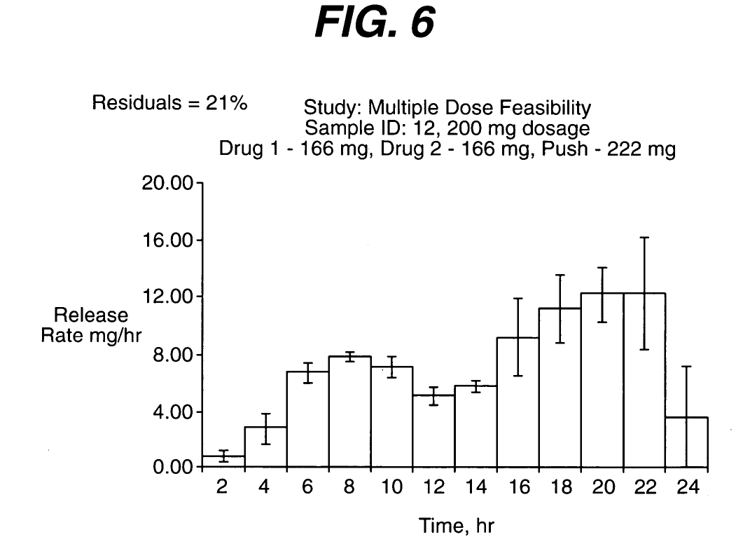

The system diagram is shown in FIG. 2. System performance is shown in FIG. 6.

### EXAMPLE 4

Topiramate Capsule Shaped Trilayer 340 mg System for Stepwise Delivery

A dosage form adapted, designed and shaped as an osmotic drug delivery device is manufactured as follows.

Preparation of First Drug Layer Granulation

80.0 g of topiramate, 10.0 g of polyethylene oxide with average molecular weight of 200,000, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of Second Drug Layer Granulation

Second, 90.0 g of topiramate, 5.0 g of cross-linked povidone with average molecular weight of more than 1,000,000(PVP XL), and 4.0 g of of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container, 0.05 g of butylated hydroxytoluene is added as an antioxidant and the resulting granulation is then lubricated with 0.5 g of stearic acid and 0.5 g of magnesium stearate.

Preparation of the Osmotic Push Layer Granulation

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000, molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Trilayer Core Compression

Next, the topiramate first drug composition, topiramate second drug composition and the push composition are compressed into tri-layer tablets on the Carver Tablet Press. First, 200 mg of the topiramate first drug composition is added to the die cavity and pre-compressed, then 200 mg of the topiramate second drug composition is added to the die cavity and pre-compressed, finally 222 mg of the push composition is added and the layers are pressed under a pressure head of approximately ¼ a metric ton into a 9/32″ (0.714 cm) diameter tri-layer longitudinal arrangement.

Preparation of the Subcoat Solution and Subcoated System

The tri-layered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the tri-layered arrangements in a pan coater until approximately 20 mg of laminate is applied to each tablet.

Preparation of the Rate Controlling Membrane and Membrane Coated System

The tri-layered subcoated cores are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the subcoated tri-layered arrangements in a pan coater until approximately 55 mg of membrane is applied to each tablet.

Drilling of Membrane Coated System

Next, a 190 mil (4.8 mm) exit passageway is drilled through the semi-permeable wall to connect the drug layer with the exterior of the dosage system. The residual solvent is removed by drying for 72 hours at 45° C. and 45% humidity.

Drying of Drilled Coated Systems

The dosage form produced by this manufacture is designed to deliver 340 mg of topiramate in a stepwise delivery pattern from the drug-containing core. The first drug layer contains 80% topiramate, 10% polyethylene oxide possessing a 200,000 molecular weight, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The second drug layer contains 90% topiramate, 5% of cross-linked povidone with average molecular weight of more than 1,000,000(PVP XL), and 4% of polyvinylpyrrolidone (Povidone K29-32), 0.05% butylated hydroxytoluene, 0.5% of magnesium stearate and 0.5% stearic acid. The push composition is comprised 64.3% polyethylene oxide comprising a 7,000,000, molecular weight, 30% sodium chloride, 5% polyvinylpyrrolidone possessing an average molecular weight of 40,000, 0.4% ferric oxide, 0.05% butylated hydroxytoluene, and 0.25% stearic acid. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% polyethylene glycol. The dosage form contains one passageway, 145 mils (3.7 mm) on the center of the drug side.

### EXAMPLE 6

The release rate of drug from devices containing the dosage forms of the invention is determined in the following standardized assay. The method involves releasing systems into acidified water (pH 3). Aliquots of sample release rate solutions are injected onto a chromatographic system to quantify the amount of drug released during specified test intervals. Drug is resolved on a C₁₈ column and detected by UV absorption. Quantitation is performed by linear regression analysis of peak areas from a standard curve containing at least five standard points.

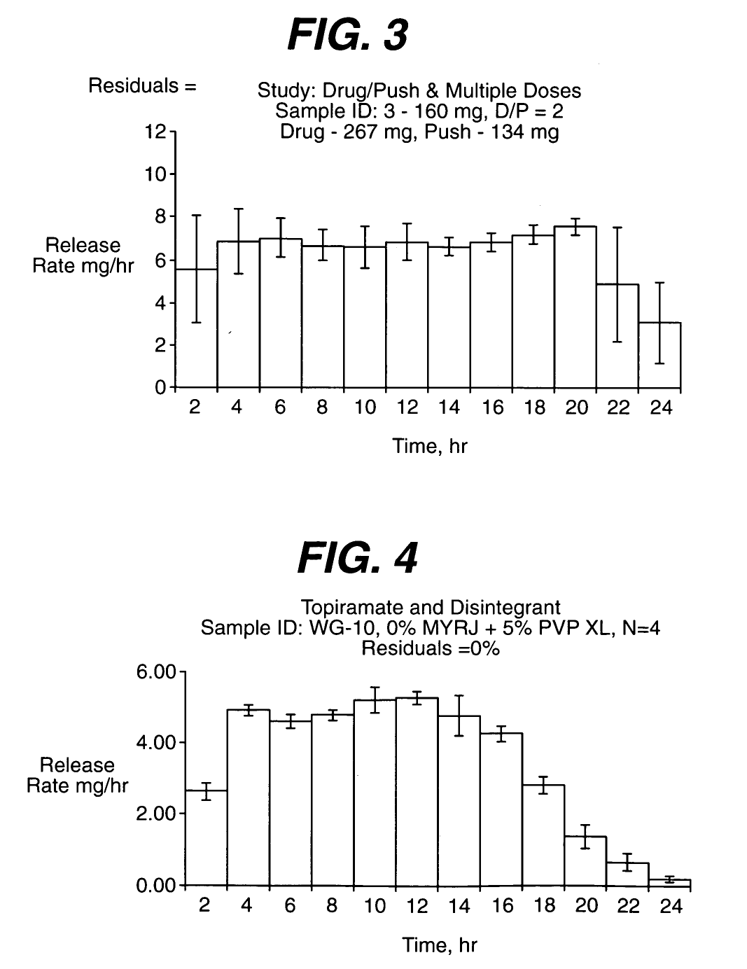

Samples are prepared with the use of a USP Type 7 Interval Release Apparatus. Each system (invention device) to be tested is weighed. Then, each system is glued to a plastic rod having a sharpened end, and each rod is attached to a release rate dipper arm. Each release rate dipper arm is affixed to an up/down reciprocating shaker (USP Type 7 Interval Release Apparatus), operating at amplitude of about 3 cm and 2 to 4 seconds per cycle. The rod ends with the attached systems are continually immersed in 50 ml calibrated test tubes containing 50 ml of acidified H₂O (acidified to pH 3.00±0.05 with phosphoric acid), equilibrated in a constant temperature water bath controlled at 37° C.±0.5° C. At the end of each time interval specified, typically one hour or two hours, the systems are transferred to the next row of test tubes containing fresh acidified water. The process is repeated for the desired number of intervals until release is complete. Then the solution tubes containing released drug are removed and allowed to cool to room temperature. After cooling, each tube is filled to the 50 ml mark with acidified water, each of the solutions is mixed thoroughly, and then transferred to sample vials for analysis by high pressure liquid chromatography (“HPLC”). Standard solutions of drug are prepared in concentration increments encompassing the range of 5 micrograms to about 400 micrograms and analyzed by HPLC. A standard concentration curve is constructed using linear regression analysis. Samples of drug obtained from the release test are analyzed by HPLC and concentration of drug is determined by linear regression analysis. The amount of drug released in each release interval is calculated. The results for various dosage forms of the invention are illustrated in FIGS. 3, 4 and 5.

1. A controlled release dosage form comprising a compound, characterized by having a high dosage, low solubility and poor dissolution rate or a pharmaceutically acceptable acid addition salt thereof, a disintegrant and no surfactant adapted to release as an erodible solid over a prolonged period of time at a stepwise, increasing rate.

2. The dosage form of claim 1 wherein the compound is topiramate.

3. The dosage form of claim 1 wherein the prolonged period of time is six hours or greater.

4. The dosage form of claim 1 wherein the prolonged period of time is eight hours or greater.

5. The dosage form of claim 1 wherein the prolonged period of time is 10 hours or greater.

6. A bioerodible composition comprising a compound characterized by having a high dosage, low solubility and poor dissolution rate or a pharmaceutically acceptable acid addition salt thereof adapted to release the compound over a prolonged period of time at a stepwise, increasing rate of release with no surfactant.

7. The composition of claim 6 wherein the compound is topiramate.

8. The composition of claim 7 further comprising polyethylene oxide and polyvinylpyrrolidone.

9. The composition of claim 8 wherein the prolonged period of time is six hours or greater.

10. The composition of claim 6 further comprising a hydrophilic polymer carrier.

11. The composition of claim 6 further comprising a disintegrant.

12. The composition of claim 10 further comprising a disintegrant.

13. A method of treating a condition in a subject responsive to administration of a compound characterized by having a high dosage, low solubility and poor dissolution rate or a pharmaceutically acceptable acid addition salt thereof which comprises orally administering to the subject a dosage form adapted to release the compound at a stepwise increasing rate of release over a prolonged period of time with no surfactant.

14. The method of claim 13 wherein the compound is topiramate.

15. The method of claim 14 wherein the dosage form contains between 50 and 1200 mg of the compound.

16. The method of claim 15 wherein the dosage form comprises an osmotic material.

17. A dosage form comprising:
a) a wall defining a compartment, at least a portion of the wall being semipermeable;
b) an exit orifice formed or formable in the wall; and
c) an expandable layer located within the compartment remote from the exit orifice and in fluid communication with the semipermeable portion of the wall; and
d) a drug layer located within the compartment adjacent the exit orifice, the drug layer comprising a compound characterized by having a high dosage, low solubility and poor dissolution rate or a pharmaceutically acceptable acid addition salt thereof with no surfactant.

18. The dosage form of claim 17 wherein the compound is topiramate.

19. The dosage form of claim 17 further comprising a flow-promoting layer between the wall and the drug layer.

20. A method of treating a condition responsive to administration of a compound comprising administering to a subject a compound characterized by having a high dosage, low solubility and poor dissolution rate or a pharmaceutically acceptable acid addition salt thereof with no surfactant which comprises maintaining over a prolonged period of time a steady state concentration of compound in the plasma of a subject between 5 ng/ml and 2500 ng/ml, wherein the quotient formed from [Cₘₐₓ−Cₘᵢₙ]/C_{avg} is 3 or less.

21. The method of claim 20 wherein the compound is topiramate.

22. The method of claim 20 wherein the quotient is 2 or less.

23. The method of claim 20 wherein the quotient is 1 or less.

24. A controlled release oral dosage form of topiramate for once-a-day
administration to a subject comprising:
(a) A core comprising:
i. Topiramate;
ii. a structural polymer;
iii. a disintegrant;
iv. no surfactant;
(b) a semipermeable membrane at least partially surrounding the core; and
(c) an exit orifice through the semipermeable membrane which communicates with the core so as to allow release of the topiramate to the environment;
wherein the dosage form releases the topiramate over a prolonged period of time at a stepwise increasing rate of release.

25. The controlled release oral dosage form of claim 24 characterized by producing a substantially ascending blood plasma concentration of topiramate in the subject after a single dose over 24 hours.

26. A method for treating a condition responsive to topiramate comprising orally administering once a day to a subject a capsule shaped tablet core dosage form containing topiramate, a disintegrant and a pharmaceutically acceptable structural polymer carrier wherein the dosage form releases the topiramate at a substantially ascending release rate for a prolonged period of time.

27. A capsule shaped tablet dosage form comprising a drug composition containing topiramate, a structural polymer carrier and a disintegrant wherein the dosage form, following oral administration to a subject, releases the active agent from the dosage form at a substantially ascending release rate for a prolonged period of time.

28. The dosage form according to claim 27 comprising:
(a) a capsule shaped tablet core containing a plurality of layers wherein the drug composition is contained in at least one layer and at least one other layer comprises a suitable fluid-expandable polymer;
(b) a semipermeable membrane surrounding the capsule shaped tablet core to form a compartment having an osmotic gradient to drive fluid from an external fluid environment contacting the semipermeable membrane into the compartment; and
(c) an orifice formed through the semipermeable membrane and into the capsule shaped tablet core to permit topiramate to be released from within the compartment into the external fluid environment.

29. The dosage form according to claim 28, wherein the capsule shaped tablet core comprises three layers and a portion of the topiramate drug composition is contained within a first layer and the remaining portion of the topiramate drug composition is contained within a second layer, wherein the portion of topiramate contained within the first layer is less than the portion of topiramate contained within the second layer, and wherein the fluid-expandable polymer is contained within a third layer and the orifice is formed through the semipermeable membrane adjacent the first layer.

30. A method for treating a condition responsive to topiramate comprising administering once a day to a subject the capsule shaped tablet dosage form of claim 29.
